(19) United States
(12) Patent Application Publication
(10) Pub. No.: US 2022/0313896 A1
(43) Pub. Date: Oct. 6, 2022

(54) DEVICE FOR REMOVING SPUTUM SCABS OF RESPIRATORY TRACT

(71) Applicant: West China Hospital of Sichuan University, Chengdu (CN)

(72) Inventor: Xiang TONG, Chengdu (CN)

(73) Assignee: West China Hospital of Sichuan University, Chengdu (CN)

(21) Appl. No.: 17/220,940

(22) Filed: Apr. 2, 2021

(51) Int. Cl. A61M 3/02; A61M 13/00

(52) U.S. Cl. CPC A61M 3/0283; A61M 13/003; A61M 25/007

(57) ABSTRACT

The present invention discloses a device for removing sputum scabs of a respiratory tract, comprising an outer housing. A fluid hole is designed on a removing part at the front end of the outer housing, and the fluid hole is respectively communicated with a liquid pump and an air pump, so that the device can be extended into the lower respiratory tract of the patient during use. Then, the sputum scabs attached to the inner wall of the respiratory tract can be softened and removed by alternately spraying liquid and gas from the fluid hole, and the removed sputum scabs can be sucked out through negative pressure holes, so as to prevent the scabs of the secretions of the lower respiratory tract from blocking the tracheae.

### TECHNICAL FIELD

The present invention relates to a respiratory medical appliance, and more particularly to a device for removing sputum scabs of a respiratory tract.

### BACKGROUND

Tracheal intubation is a clinical operation for endotracheal anesthesia and rescue of patients, and also the most reliable means to keep the upper respiratory tract unobstructed. In clinical medical treatment, the obstruction of the upper respiratory tract can be removed urgently; the secretions of the lower respiratory tract can be absorbed; and a respiratory passage can be established for the patient. In implementation of the tracheal intubation, the trachea of the patient is often blocked because the secretions of the lower respiratory tract scab, and the secretions need to be removed in time. However, at this moment, due to tracheal intubation, the water in the secretions of the respiratory tract evaporates quickly, Thick sputa are easily formed and attached to the tracheal wall, and are extremely difficult to be removed, thereby bringing pain to the patient. Therefore, it is necessary to study and improve the structure of sputum scab removing instruments.

### SUMMARY

A purpose of the present invention is to provide a device for removing sputum scabs of a respiratory tract with respect to the above defects, to solve the technical problems in the prior art that in implementation of the tracheal intubation, sputum scabs are easily formed on the lower respiratory tract of the patient and difficult to be removed, thereby bringing pain to the patient.

To solve the above technical problems, the present invention adopts the following technical solutions:

A device for removing sputum scabs of a respiratory tract provided by the present invention comprises an outer housing. The front end of the outer housing is provided with a removing part; the front end of the removing part is provided with a fluid hole; the fluid hole is respectively communicated with an air flow pipe and a liquid flow pipe through pipelines; the air flow pipe and the liquid flow pipe extend to the outside of the outer housing; the liquid flow pipe is communicated with a liquid pump; the air flow pipe is communicated with an air pump; the outer edge of the removing part is provided with a plurality of negative pressure holes; the negative pressure holes are communicated with a negative pressure cavity through a negative pressure pipeline inside the outer housing; and the outer edge of the removing part forms a circular arc with the front end.

Preferably, a further technical solution is as follows: the fluid hole is communicated with the external air pump through the pipeline and the air flow pipe; the fluid hole is communicated with the external liquid pump through the pipeline and the liquid flow pipe; and the liquid pump and the air pump are used for alternately spraying liquid and gas through the fluid hole.

A further technical solution is as follows: the outer housing is a circular tubular body, and the removing part is a conical tubular body.

A further technical solution is as follows: the outer edge of the removing part is sleeved with an annular friction sleeve, and the annular friction sleeve is provided with openings corresponding to the negative pressure holes.

A further technical solution is as follows: the plurality of the negative pressure holes are distributed in a cross array on the outer edge of the removing part.

A further technical solution is as follows: the front end of the removing part is provided with an illuminating lamp; the illuminating lamp is connected to a power supply; and the power supply is installed in the outer housing.

A further technical solution is as follows: the front end of the removing part is provided with a camera; a lamp of the camera is connected to a control module; the control module is installed in the outer housing; and the control module is also connected to the power supply.

Compared with the prior art, the present invention has the following beneficial effects: the fluid hole is designed on the removing part at the front end of the outer housing, and the fluid hole is respectively communicated with the liquid pump and the air pump, so that the device can be extended into the lower respiratory tract of the patient during use. Then, the sputum scabs attached to the inner wall of the respiratory tract can be softened and removed by alternately spraying the liquid and the gas through the fluid hole, and the removed sputum scabs can be sucked out through the negative pressure holes, so as to prevent the scabs of the secretions of the lower respiratory tract from blocking the tracheae, so that the pain of the patient is reduced during implementation of the tracheal intubation. Meanwhile, the device for removing sputum scabs of the respiratory tract provided by the present invention has simple structure, and is suitable for industrial production and easy for popularization.

### BRIEF DESCRIPTION OF THE DRAWINGS

FIG. 1 is a structural schematic diagram of an embodiment of the present invention;

FIG. 2 is a top structural schematic diagram of a removing part in FIG. 1;

FIG. 3 is a structural schematic diagram of a second embodiment of the present invention;

FIG. 4 is a structural schematic diagram of a third embodiment of the present invention; and

FIG. 5 is a structural schematic diagram of a fourth embodiment of the present invention.

In the drawings, 1 outer housing; 2 removing part; 3 fluid hole; 4 air flow pipe; 5 liquid flow pipe; 6 liquid pump; 7 air pump; 8 negative pressure hole; 9 negative pressure cavity; 10 annular friction sleeve; 11 camera; 12 illuminating lamp.

### DETAILED DESCRIPTION OF THE EMBODIMENTS

The present invention is further illustrated below in combination with the drawings.

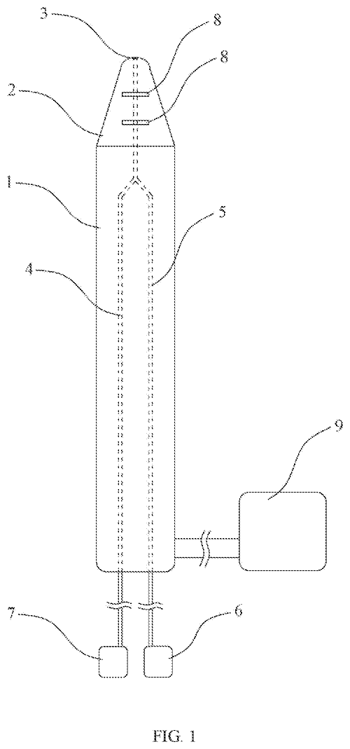

As shown in FIG. 1, an embodiment of the present invention is a device for removing sputum scabs of a respiratory tract, comprising an outer housing 1. The front end of the outer housing 1 is provided with a removing part 2; the removing part 2 can come into contact with the lower respiratory tract of a human body; meanwhile, a fluid hole 3 is designed on the front end of the removing part 2; the fluid hole 3 can be used for spraying fluid of gas and liquid; and under the action of a power pump, the fluid can be sprayed out from the fluid hole 3 at certain pressure. Based on the above purpose, the fluid hole 3 needs to be communicated with an air flow pipe 4 and a liquid flow pipe 5 respectively through pipelines; accordingly, the air flow pipe 4 and the liquid flow pipe 5 need to extend to the outside of the outer housing 1; the liquid flow pipe 5 is communicated with a liquid pump 6; and the air flow pipe 4 is communicated with an air pump 7. More importantly, the outer edge of the removing part 2 needs to be provided with a plurality of negative pressure holes 8; and the negative pressure holes 8 are communicated with a negative pressure cavity 9 through a negative pressure pipeline (not shown in the figures) inside the outer housing 1. Herein, in order to save the space in the outer housing 1, the negative pressure pipeline can be designed outside the air flow pipe 4 and the liquid flow pipe 5. Moreover, the above negative pressure pipeline also needs to be communicated with a sputum collecting tank; and a check valve needs to be installed between the sputum collecting tank and the negative pressure pipeline to ensure that after entering the sputum collecting tank, the sputa are not sucked out by negative pressure in the negative pressure pipeline. Meanwhile, to facilitate the contact between the removing part 2 and the inner wall of the lower respiratory tract of the human body, the outer edge and the front end of the removing part 2 can be designed as circular arcs to reduce the irritation and discomfort to the inner wall of the respiratory tract of the human body caused by the removing part 2.

Herein, preferably, based on the device of the above structure, the fluid hole 3 is directly communicated with the external air pump 7 through the pipeline and the air flow pipe 4; and the fluid hole 3 is communicated with the external liquid pump 6 through the pipeline and the liquid flow pipe 5. Thus, under the action of the liquid pump 6 and the air pump 7, liquid and gas can be alternately sprayed out through the fluid hole 3, and the output pressure of the liquid pump 6 and the air pump 7 is adjusted according to required pressure. Meanwhile, as shown in FIG. 1, in the shape design of the device, the outer housing 1 can be designed as a circular tubular body, and the removing part 2 can be designed as a conical tubular body.

In the present embodiment, the fluid hole 3 is designed on the removing part 2 at the front end of the outer housing 1, and the fluid hole 3 is respectively communicated with the liquid pump 6 and the air pump 7, so that the device can be extended into the lower respiratory tract of the patient during use. Then, the sputum scabs attached to the inner wall of the respiratory tract can be softened and removed by alternately spraying the liquid and the gas through the fluid hole, and the removed sputum scabs can be sucked out through the negative pressure holes, so as to prevent the scabs of the secretions of the lower respiratory tract from blocking the tracheae, so that the pain of the patient is reduced during implementation of the tracheal intubation.

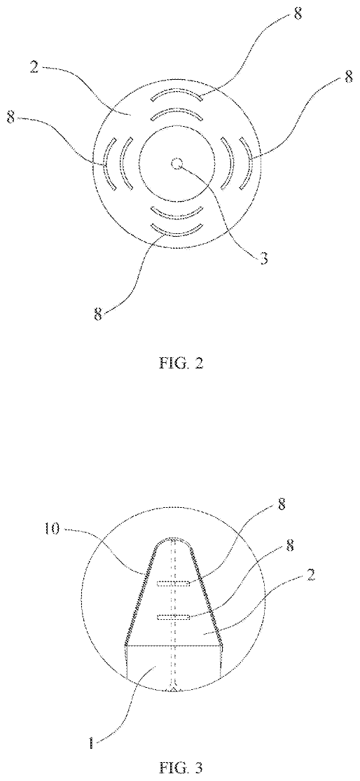

According to another embodiment of the present invention, as shown in FIG. 2, for the convenience of sucking the secretions of the sputa on the lower respiratory tract, the plurality of the negative pressure holes S are designed to be distributed in a cross array on the outer edge of the removing part 2. In the present embodiment, preferably, to further reduce the irritation to the inner wall of the lower respiratory tract by the removing part 2 and avoid cross infection during the use of the device, as shown in FIG. 3, the outer edge of the removing part 2 is sleeved with an annular friction sleeve 10; and the annular friction sleeve 10 is disposable and can be processed by soft silica gel. Meanwhile, to avoid influencing the negative pressure, openings corresponding to the negative pressure holes 8 shall be added on the annular friction sleeve 10 to ensure that the negative pressure holes 8 can be exposed and the secretions on the inner wall of the lower respiratory tract can be normally sucked out.

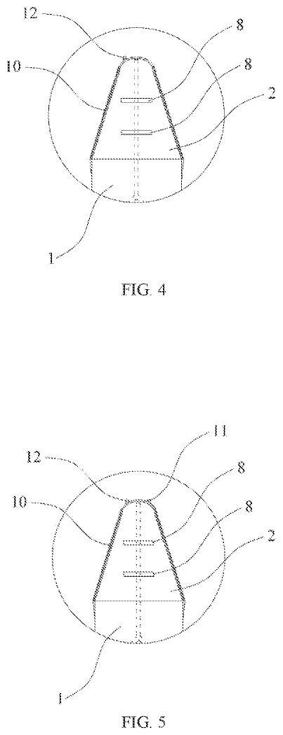

Based on the above structure, during the test, the inventor also finds that the light of the lower respiratory tract of the human body is insufficient, and the operation of removing the sputum scabs is not accurate enough. Therefore, the device in the above embodiment is further improved. As shown in FIG. 4, the inventor firstly thinks of directly installing an illuminating lamp 12 on the front end of the removing part 2, connecting the illuminating lamp 12 to a power supply and then installing the power supply in the outer housing 1. Thus, during use, the illuminating lamp 12 is lit to illuminate the inner wall of the lower respiratory tract of the human body, which is conducive to improving the operation accuracy of doctors when the sputum scabs are removed. On this basis, the inventor also learns from the imaging technology of a miniature camera. As shown in FIG. 5, a miniature camera 11 is installed on the front end of the removing part 2; a lamp of the camera 11 is connected to a control module; the control module is installed in the outer housing 1; and the control module is also connected to the power supply. Thus, a real-time image of the inner wall of the lower respiratory tract of the human body can be collected through the camera 11, and the real-time image is projected onto an external display screen through the control module, to assist the doctors to remove the sputum scabs on the lower respiratory tract of the patient more accurately. The operation is more convenient, and conducive to increasing the efficiency of removing the sputum scabs. Because the miniature camera and the image transmission technology have been maturely used in the existing medical equipment, the specific imaging principle of the image is not detailed herein.

As shown in FIG. 1 to FIG. 5, in actual use of a preferred embodiment of the present invention, when the sputum scabs on the lower respiratory tract of the patient need to be removed for the tracheal intubation or other clinical treatment operations, an annular friction sleeve 10 made of silica gel is sleeved on the removing part 2. The external liquid pump 6 is started at first; the pressure is adjusted and then distilled water is sprayed out from the fluid hole 3 through the liquid flow pipe 5 by the liquid pump 6 and acts on the sputum scabs of the inner wall of the respiratory tract to soften the sputum scabs. Then, the external air pump 7 is started; the pressure is adjusted and then air is sprayed out from the fluid hole 3 through the air flow pipe 4 by the air pump 7. The softened sputum scabs are washed down through the air flow. Meanwhile, the negative pressure cavity 9 forms negative pressure in the negative pressure pipeline, so that the washed sputum scabs are immediately sucked into the negative pressure holes 8 and finally enter the sputum collecting tank. In the above process, the illuminating lamp 12 and the camera 11 at the front end of the removing part 2 can also be used to assist in finding the positions of the sputum scabs on the inner wall of the lower respiratory tract of the patient more accurately to ensure the removing efficiency, facilitate the successful implementation of the tracheal intubation and other related clinical treatment operations and reduce the pain of the patient.

“One embodiment”, “another embodiment”, “embodiment”, etc. mentioned in this description mean that specific features, structures or characteristics illustrated in combination with the embodiment are included in at least one embodiment which is generally illustrated in the present application. The same statements which appear in multiple places in this description do not necessarily refer to the same embodiment. Further, when a specific feature, structure or characteristic is described in combination with any embodiment, it is intended that the realization of the feature, structure or characteristic in combination with other embodiments also falls within the scope of the present invention.

Although the present invention is described herein by referring to multiple explanatory embodiments of the present invention, it shall be understood that those skilled in the art can design many other modifications and implementations, and these modifications and implementations will fall within the principle scope and spirit disclosed by the present application. More specifically, various variations and improvements may be made to the components of the theme combination layout and/or the layout within the scope of the disclosure, drawings and claims of the present application. In addition to the variations and improvements to the components and/or layout, other applications will also be apparent to those skilled in the art.

What is claimed is:

1. A device for removing sputum scabs of a respiratory tract, comprising an outer housing (1), wherein the front end of the outer housing (1) is provided with a removing part (2); the front end of the removing part (2) is provided with a fluid hole (3); the fluid hole (3) is respectively communicated with an air flow pipe (4) and a liquid flow pipe (5) through pipelines; the air flow pipe (4) and the liquid flow pipe (5) extend to the outside of the outer housing (1); the liquid flow pipe (5) is communicated with a liquid pump (6); the air flow pipe (4) is communicated with an air pump (7); the outer edge of the removing part (2) is provided with a plurality of negative pressure holes (8); the negative pressure holes (8) are communicated with a negative pressure cavity (9) through a negative pressure pipeline inside the outer housing (1); and the outer edge of the removing part (2) forms a circular arc with the front end.

2. The device for removing sputum scabs of the respiratory tract according to claim 1, wherein the fluid hole (3) is communicated with the external air pump (7) through the pipeline and the air flow pipe (4); the fluid hole (3) is communicated with the external liquid pump (6) through the pipeline and the liquid flow pipe (5); and the liquid pump (6) and the air pump (7) are used for alternately spraying liquid and gas through the fluid hole (3).

3. The device for removing sputum scabs of the respiratory tract according to claim 1, wherein the outer housing (1) is a circular tubular body, and the removing part (2) is a conical tubular body.

4. The device for removing sputum scabs of the respiratory tract according to claim 1, wherein the outer edge of the removing part (2) is sleeved with an annular friction sleeve (10), and the annular friction sleeve (10) is provided with openings corresponding to the negative pressure holes (8).

5. The device for removing sputum scabs of the respiratory tract according to claim 4, wherein the plurality of the negative pressure holes (8) are distributed in a cross array on the outer edge of the removing part (2).

6. The device for removing sputum scabs of the respiratory tract according to claim 1, wherein the plurality of the negative pressure holes (8) are distributed in a cross array on the outer edge of the removing part (2).

7. The device for removing sputum scabs of the respiratory tract according to claim 1, wherein the front end of the removing part (2) is provided with an illuminating lamp (12); the illuminating lamp (12) is connected to a power supply; and the power supply is installed in the outer housing (1).

8. The device for removing sputum scabs of the respiratory tract according to claim 7, wherein the front end of the removing part (2) is provided with a camera (11); a lamp of the camera (11) is connected to a control module; the control module is installed in the outer housing (1); and the control module is also connected to the power supply.

9. The device for removing sputum scabs of the respiratory tract according to claim 1, wherein the front end of the removing part (2) is provided with a camera (11); a lamp of the camera (11) is connected to a control module; the control module is installed in the outer housing (1); and the control module is also connected to the power supply.
